(19) United States
(12) Patent Application Publication
(10) Pub. No.: US 2013/0102986 A1
(43) Pub. Date: Apr. 25, 2013

(54) Wearable Absorbent Articles With Reusable Chassis Having Extensible Body Zones

(71) Applicant: The Procter & Gamble Company, Cincinnati, OH (US)

(72) Inventors: Oscar Antonio Ruiz, Mason, OH (US); Donald Carroll Roe, West Chester, OH (US)

(73) Assignee: The Procter & Gamble Company, Cincinnati, OH (US)

(21) Appl. No.: 13/655,828

(22) Filed: Oct. 19, 2012

Related U.S. Application Data
(60) Provisional application No. 61/548,775, filed on Oct. 19, 2011.

(51) Int. Cl. A61F 13/505

(52) U.S. Cl. 604/385.14

(57) ABSTRACT

Reusable chassis with body zones having lateral extensibilities that are low enough to at least reduce sag but high enough to accommodate swelling by absorbent inserts.

### BACKGROUND

Wearable absorbent articles include diapers and incontinence undergarments. A wearable absorbent article can receive and contain bodily exudates while being worn by a wearer. Some wearable absorbent articles have reusable chassis and absorbent inserts, which can be disposable absorbent inserts or reusable absorbent inserts. Using such articles can reduce waste, when compared with using fully disposable absorbent articles.

However, some wearable absorbent article can perform poorly if the body zone of the reusable chassis is not configured with a proper lateral extensibility. If the body zone has a lateral extensibility that is too high, then, as the article receives bodily exudates, portions of the chassis may tend to sag, causing the article to look unattractive. If the body zone has a lateral extensibility that is too low, then, as the article receives bodily exudates, the chassis may not accommodate swelling by the absorbent insert, causing the article to leak.

### BRIEF DESCRIPTION OF THE DRAWINGS

FIG. 1 is a perspective view of a wearable absorbent article as it might appear being worn by a wearer about the lower torso;

FIG. 2A is a plan view of a chassis opened and laid flat, outer surface facing the viewer;

FIG. 2B is a plan view of a chassis opened and laid flat, inner surface facing the viewer;

FIG. 3 is a perspective view of a disposable absorbent insert shown apart from a chassis, as it might appear in a free-standing, relaxed state;

FIG. 4 is a plan view of a disposable absorbent insert shown stretched out and laid flat, body-facing surfaces facing the viewer;

FIG. 5A is across sectional view of an example of an insert such as shown in FIG. 4, taken line 5A-5A in FIG. 4;

FIG. 5B is across sectional view of an example of a insert such as shown in FIG. 4, taken along line 5B-5B in FIG. 4;

FIG. 5C is across sectional view of another example of an insert such as shown in FIG. 4, taken along line 5C-5C in FIG. 4;

FIG. 5D is a cross sectional view of another example of an insert such as shown in FIG. 4, taken along line 5D-5D in FIG. 4;

FIG. 5E is a cross sectional view of another example of an insert such as shown in FIG. 4, taken along line 5E-5E in FIG. 4; and

FIG. 5F is a cross sectional view of another example of an insert such as shown in FIG. 4, taken along line 5F-5F in FIG. 4.

### DETAILED DESCRIPTION OF THE INVENTION

Definitions

For purposes of this description, the following terms have the meanings set forth:

“Absorbent insert” means a component of a wearable absorbent article that is adapted to contain and/or absorb urine, feces, menses or any combination thereof, and is adapted to be installable and removable as a modular unit, from a chassis.

“Chassis” means a component of a wearable absorbent article that is adapted to be worn about the lower torso of a wearer, and is adapted to support an absorbent insert and hold the insert next to the wearer's body.

“Disposable”, when referring to an absorbent insert, means that the absorbent insert is not adapted or intended to be effectively sanitarily laundered in an ordinary household laundering process and ordinary household equipment, and thereby is ordinarily unsuitable for sanitary and effective reuse so as to provide as-new intended functions and performance, following soiling by exudates and removal from a chassis. By way of non-limiting examples, effective laundering may be frustrated or prevented, causing the insert to be disposable, by inclusion of materials and/or construction: that do not retain their substantial as-new physical shape or structure through ordinary household laundering and drying so as to be effective as-new in reuse; that absorb aqueous liquids and cannot be sufficiently dried/dehydrated in ordinary household drying equipment and ordinary drying cycles so as to he effective as-new in reuse; that dissolve or substantially degrade in ordinary household laundering or drying, causing the insert to be substantially damaged or rendered useless; and/or that cannot be effectively cleaned of exudate material through ordinary laundering, so as to be sanitary and otherwise acceptable for re-use.

“Fastener component” means any component of a system that effects removable fastening, attachment or holding of a first structure to a second structure. The system may have a single fastener component, for example, an adhesive patch on the first structure adapted to adhere to one or more types of surfaces on the second structure, or a hook, or patch of hooks on the first structure, adapted to catch on one or more types of surfaces on the second structure. By way of further example, any structure such as a pocket, strap, hook, buckle, etc. on a first structure adapted to capture and retain, in whole or in part, the second structure, is a “fastener component” as used herein. The system also may comprise two or more fastener components, for example, respective components of a hook-and-loop fastening system (such as VELCRO), respective surfaces having a cohesive material applied thereto; male and female snap fastener components, a button and button hole, slot or loop, other fastenably cooperating elements, etc. Other examples of fastener components include zipper components, “zip lock” engaging components, loops, posts, pockets, bands or straps, microfasteners, macrofasteners, and fastener components such as described in U.S. Pat. Nos. 6,936,039; 6,893,388; 6,669,618; 6,432,098; and 6,251,097, and U.S. Published Applications, Pub. Nos. 2005/0234419; 2005/0215971; 2005/0215970; 2005/0130821; 2004/0023771; 2003/0233082; 2003/01.19641; 2003/0088220; and 2002/0169431.

“Lateral” (and forms thereof), with respect to a wearer, means along a direction generally transverse or across the direction extending from the front to the rear of the wearer, or vice versa. With respect to a component of a wearable absorbent article, “lateral” (and forms thereof), means along a direction generally transverse or across the direction extending along the component as it would be properly situated on a wearer, from the front to the rear of the wearer, or vice versa.

“Longitudinal” (and forms thereof), with respect to a wearer, means along a direction generally extending from the front to the rear of the wearer, or vice versa. With respect to a component of a wearable absorbent article, “longitudinal” (and forms thereof), means along a direction generally extending along the component as it would be properly situated on a wearer, from the front to the rear of the wearer, or vice versa.

“Reusable”, when referring to a chassis, means a chassis that is adapted to permit removal of at least a first insert, and replacement thereof with at least a second insert, without substantial destruction of any components of the chassis that are necessary to provide the substantial as-new functionality of the chassis, and without the necessity of any repair or reconstruction following such insert replacement.

“Use,” with respect to a chassis, means one event of the wearing of the chassis until the time an absorbent insert is replaced.

“User” means a caregiver or other person who may apply a wearable absorbent article to a wearer. Where the wearer is capable of donning the wearable absorbent article him/herself, the wearer is also a “user”.

“Wearer” means a person who may wear a wearable absorbent article as described herein.

“Wearable absorbent article” means any article designed to be worn about the lower torso and to contain and/or absorb urine, feces, menses or any combination thereof. “Wearable absorbent article” includes but is not limited to baby or children's diapers (of the “tape”-fastenable, otherwise fastenable, “pull-up” or any other variety), training pants and adult incontinence pants, briefs and the like.

Two-Piece Wearable Absorbent Articles

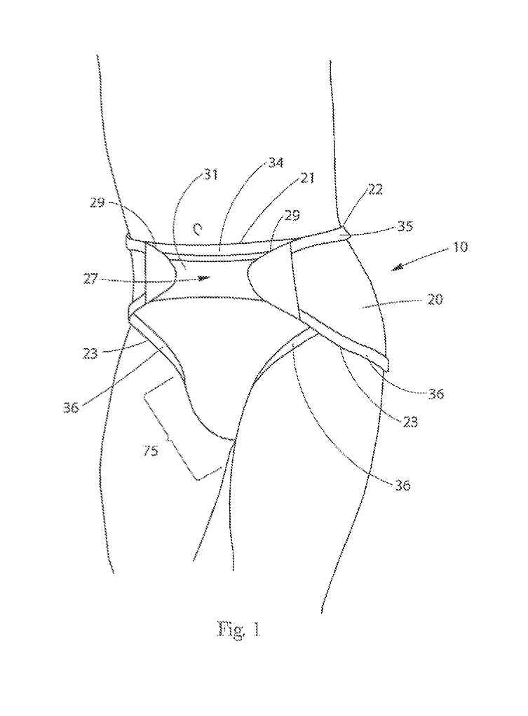

FIG. 1 depicts an example of a wearable absorbent article 10 having certain features, as it might appear while being worn by a wearer. Wearable absorbent article 10 may include and outer chassis 20. having front waist edge 21, rear waist edge 22, and a pair of leg opening edges 23.

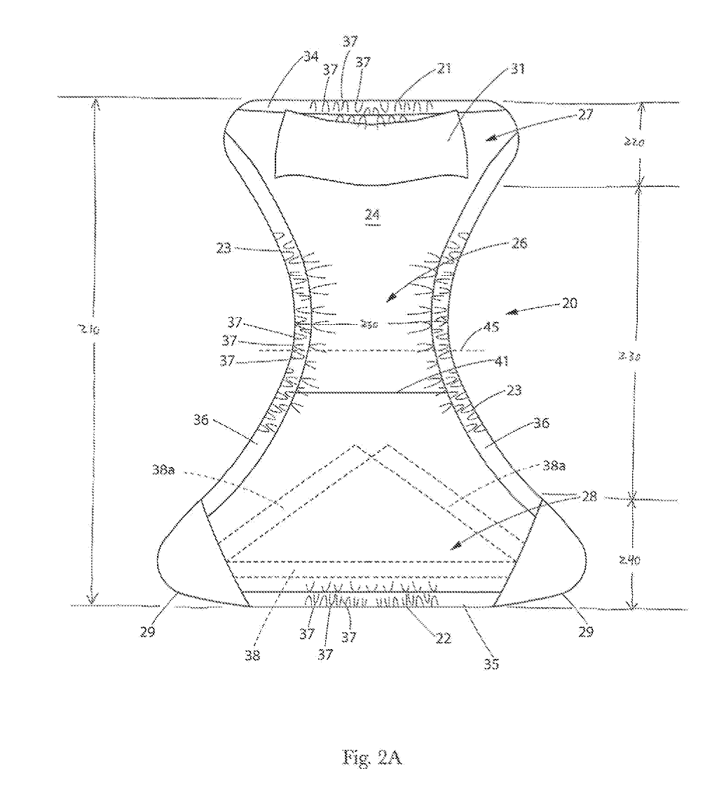

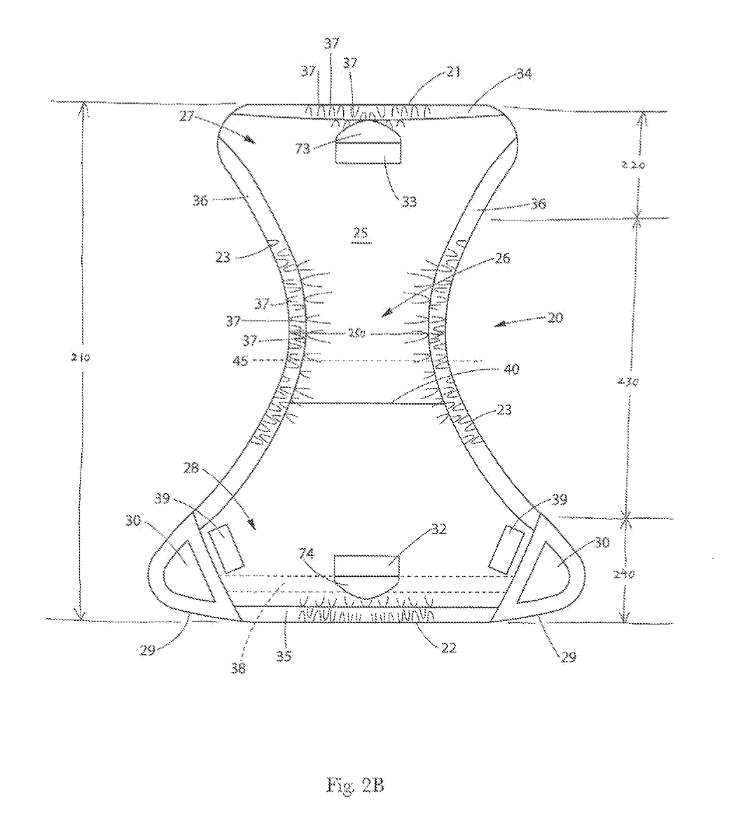

FIGS. 2A and 213 depict a chassis 20 as it may appear opened and laid flat. In FIG. 2A, the outer surfaces of chassis 20 face the viewer; in FIG. 2B, the inner surfaces of chassis 20 face the viewer. Front and rear waist edges 21, 22 are depicted at the top and bottom of the drawings, respectively. Chassis 20 may have crotch region 26, front region 27, rear region 28 and a pair of fastening ears 29 laterally extending from rear region 28. Chassis 20 will have a length from the fowardmost portion of front waist edge 21 to the rearwardmost portion of rear waist edge 22, and a chassis lateral axis 45 equally dividing this length. Thus, front region 27 is forward of chassis lateral axis 45, and rear region 28 is rearward of chassis lateral axis 45. Chassis 20 may have disposed thereon one or more insert fastener components such as front and rear insert fastener components 33, 32.

As shown in FIGS. 2A and 2B, the chassis 20 has a front waist zone 220, adjacent to the front waist edge 21, a rear waist zone 240, adjacent to the rear waist edge 22, and a body zone 230 disposed between the front waist zone 220 and the rear waist zone 240 and overlapping the lateral axis 45. In alternate embodiments, a front waist zone may be proximate to a front waist edge and/or a back waist zone may be proximate to a back waist edge.

All of the measurements described below are made when the chassis is laid out flat. The chassis 20 has an overall longitudinal length 210, measured longitudinally between the front waist edge 21 and the rear waist edge 22. The front waist zone 220 has an overall longitudinal length, measured longitudinally between the front waist edge 21 and a longitudinally innermost extent of the front waist zone 220. The rear waist zone 240 has an overall longitudinal length, measured longitudinally between the rear waist edge 22 and a longitudinally innermost extent of the rear waist zone 240.

The body zone 230 is disposed longitudinally between the front waist zone 220 and the rear waist zone 240, laterally inboard to the leg bands 36, and overlapping the lateral axis 45. The body zone 230 has an overall longitudinal length, measured longitudinally between the longitudinally innermost extent of the front waist zone 220 and the longitudinally innermost extent of the rear waist zone 240. The chassis 20 also has a narrowest lateral width 250, measured laterally as the shortest distance between the leg opening edges 23. The narrowest lateral width 250 is in the body zone 230.

The body zone 230 can have an overall longitudinal length that is 30-70 percent of the overall longitudinal length 210 of the chassis, or any integer percentage between these values, or any range formed by any of these percentages. The front waist zone 220 and the back waist zone 340 can each have an overall longitudinal length that is 10-40 percent of the overall longitudinal length 210 of the chassis, or any integer percentage between these values, or any range formed by any of these percentages. The narrowest lateral width 250 can be 20-60 percent of the overall longitudinal length 210 of the chassis, or any integer percentage between these values, or any range formed by any of these percentages.

The body zone 230 can have a lateral extensibility that is 0.03-0.24 Newtons per millimeter, or any increment of 0.01 Newtons per millimeter between these values, or any range formed by any of these values. When subjected to the ear extension test, as described herein, either or both of the front waist zone 220 and the back waist zone 340 can have a lateral extensibility that is 4-16 Newtons, or any integer between these values, or any range formed by any of these values.

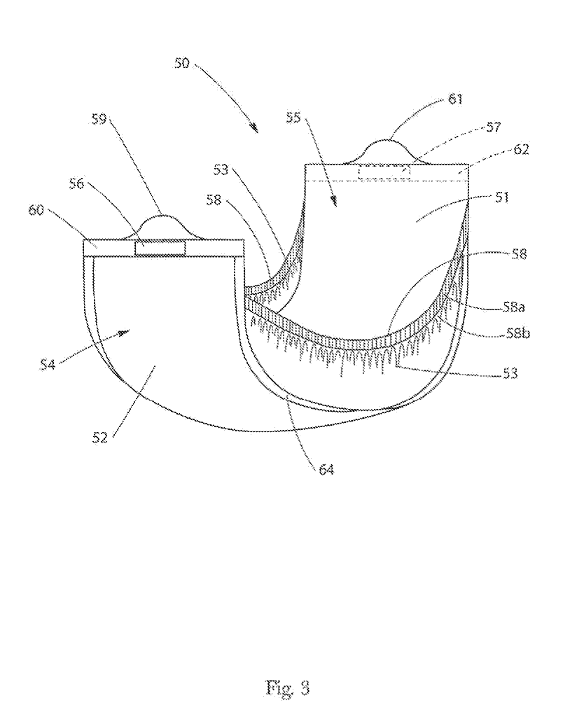

FIG. 3 depicts a disposable absorbent insert 50 that may form an inner component of the wearable absorbent article 10, shown in perspective view as it might appear in a free-standing, relaxed state, apart from chassis 20. Insert 50 may be designed to contain and/or absorb body exudates, and may be made of pliable materials as will be described further below. Insert 10 has forward region 54 and rearward region 55, and may include front fastener component 56 and rear fastener component 57. Insert 10 may include a body-facing liner or topsheet 51, outer liner or backsheet 52, and a pair of standing cuffs 53. Referring to FIG. 4, insert 50 will have a length from the forwardmost portion of forward region 54 to the rearwardmost portion of rearward region 55, and an insert lateral axis 70 equally dividing this length. Thus, forward region 54 is forward of insert lateral axis 70, and rearward region 55 is rearward of insert lateral axis 70.

Referring to FIGS. 2B and 3, insert 50 may have rear fastener component 57 disposed thereon. Alternatively, or in addition, chassis 20 may have rear insert fastener component 32 disposed thereon. Similarly, insert 50 may have front fastener component 56 disposed thereon. Alternatively, or in addition, chassis 20 may have front insert fastener component 32 disposed thereon. If a two-component fastening system is used, fastener component pairs 57, 32 and 56. 33 may be cooperating components that effect fastening therebetween when these respective components are brought together. Thus, in the example depicted, in order to install absorbent insert 50 into chassis 20, a user may lay chassis 20 flat, inner surface 25 facing up, stretch and orient insert 50 such that rear fastener component 57 faces rear insert fastener component 32 and front fastener component 56 faces front insert fastener component 33, and bring these respective fastener component pairs 57, 32 and 56, 33 together to effect fastening therebetween.

If it is desired that chassis 20 be reusable, for chassis 20 to remain substantially sanitary and useful (without requiring laundering or disposal) after removal and replacement of an insert, it may be desired that all parts of chassis 20 remain substantially unsoiled. after an exudation of waste (especially fecal matter) by the wearer. Thus, it may be desired that when insert 50 is installed within a chassis 20, there is no non-removable portion or component of chassis 20 that lies over or covers a substantial portion of wearer-facing surfaces of insert 50 (expressed differently, no non-removable portion or component of chassis 20 is situated between a substantial portion of insert 50 and the wearer when the wearable absorbent article is worn), at least in the areas proximate to wearer body features that discharge exudates. Thus, it may be desired that chassis 20 include no non-removable cover sheet or the like that covers or contains substantial portions of wearer-facing surfaces of insert 50 within chassis 20, nor any overlying structures such as pockets, straps or flaps that substantially wrap or cover the insert proximate to exudate discharge points, or lie substantially between insert 50 and the wearer's anus and/or genitals, when the wearable absorbent article is worn. If chassis 20 lacks such overlying structures, this may increase the likelihood that the wearer's exudates will contact only insert 50, and not portions of chassis 20.

Referring to FIGS. 1, 2A and 2B, it can be seen that wearable absorbent article 10 may be placed on a wearer by wrapping chassis 20 between the wearer's legs and under the buttocks such that crotch region 26 is between the wearer's legs, bringing front waist edge 21 and rear waist edge 22 into the positions approximately as shown in FIG. 1, and then securing fastening ears 29 to front region 27, thereby forming a pant-like garment about the wearer as suggested in FIG. 1. When insert 50 has been installed into chassis 20, insert 50 will then be disposed within chassis 20, next to the wearer, with the standing cuffs 53 oriented and extending longitudinally adjacent the inner portions of leg edges 23 (i.e., longitudinally between the wearer's legs).

### Examples of Possible Chassis Details

Fastening System

Referring to FIGS. 2A and 2B, to enable fastening of fastening ears 29 to front region 27, fastening ears 29 may have chassis fastener components 30 disposed thereon. Alternatively, or in addition, front region 27 may have disposed thereon one or more receiving fastener components 31 disposed thereon. Fastener components 30, 31 may be selected so as to be cooperative to effect fastening of ears 29 to front region 27.

In one example, chassis fastener components 30 may include a patch of hooks, and receiving fastener component 31 may include a patch of loops. An example of a suitable hook-and-loop fastening system is a VELCRO system (a product of Velcro Industries B.V.) A hook-and-loop fastening system provides certain advantages. Because the respective hook and loop components are supplied in sheet form, they may be cut into suitably shaped patches that can be affixed to a cloth or nonwoven substrate by various mechanisms, including adhesive bonding, mechanical bonding, ultrasonic bonding, sewing, stitching, serging, edging, and the like. If patches of hooks or loops are affixed to fastening ears 29 to form chassis fastener components 30, as suggested by FIG. 2B, a laterally extended patch of cooperating hook or loop material can be affixed to chassis front region 27 to form a receiving fastener component 31, as suggested by FIG. 2A. By providing for fastening of ears 29 to front region 27 at laterally varying locations thereon, this arrangement provides for easy and simple adjustability of waist opening size of the wearable absorbent article.

In another example, as noted above, the chassis fastening system may include other types of fastener components. To provide for waist opening size adjustability, fastening components disposed on fastening ears 29 that cooperate with extended surfaces or multiple cooperating fastening components disposed on front region 27 may be used. Receiving fastening elements may be multiply disposed on front region 27 in laterally arranged, varying locations, allowing for fastenability of respective ears 29 to front region 27 at laterally varying locations. Referring to FIG. 2B, chassis 20 also may have one or more respective fastener protectors 39 disposed thereon. This feature may prevent fastening elements having features likely to randomly and unintentionally engage and catch on portions of the chassis, or other articles, during storage, carrying, laundering and similar/related activities, from doing so, thereby avoiding potential bunching, entangling and/or damage to either chassis 20 or other articles during such activities. For example, if fastening components 30 are patches of hooks, appropriately placed fastener protectors 39 may include patches of corresponding loops. This can enable the user to holdably fold over the portions of ears 29 that include hook components, thereby sheathing them and preventing them from snagging other articles when chassis 20 is not being worn.

Materials

Chassis 20 and/or layers or portions thereof may be made of any knitted, woven or nonwoven textile or textile-like material that is appropriately compatible with skin of the intended wearer(s). Chassis 20 may be constructed. of durable and/or semi-durable materials. Generally, only for purposes of reference in this description, “durable” refers to a woven or knitted textile material of any kind that may be used as a component of a washable clothing article; and “semi-durable” refers to a nonwoven material or laminate thereof that when used as a chassis material can withstand more than one use with an insert without losing its structural integrity to an extent that renders it unserviceable. Thus, chassis 20 may be constructed of materials and construction that make it reusable and/or washable.

Durable materials of which chassis 20 may be constructed may include any natural or synthetic textile materials known in the diaper, pant, underwear, performance clothing, sport clothing, or general clothing or textile arts. Durable materials may include woven or knitted textiles made of natural fibers such as cotton, linen, wool, bamboo, hemp, silk, rayon, and the like, as well as blends of any of these fibers with any other(s), or with synthetic fibers. Examples of synthetic fibers suitable for use as components of durable materials include polyester, nylon, spandex and/or other elastomer fibers. Durable chassis materials also may include breathable water repellent materials such as GORE-TEX (a product of W. L. Gore & Associates, Inc., Elkton, Md.), fabrics comprising microencapsulated phase-change polymer materials such as OUTLAST COMFORTEMP fabrics (products of Outlast Technologies, Boulder, Colo.—see U.S. Pat. No. 6,514,362 and U.S. Pat. No. 6,207,738, for example), COOLMAX (a product of Invista, Wichita, Kans.), and the like.

Suitable durable materials may be formed in any weave or knit fabric form, including birdseye fabric, terry, fleece, flannel, knits, stretch knits, sherpa, suedecloth, microfleece, satin, velour, Burley knits, etc. Suitable examples include POLARTECH POWER DRY, POWER STRETCH and WIND PRO (products of Polartec, LLC, Lawrence, Mass.). Knitted textiles, which may be more inherently stretchable and elastic than woven or nonwoven materials, may impart better fit, comfort and/or appearance to the chassis. Incorporation of fibers of spandex or other elastomer also may also enhance stretchability and elasticity, and thereby impart better fit, comfort and/or appearance to the chassis, than textiles not including such elastomeric fibers.

Specific suitable examples for durable chassis materials include jersey knits of blends of: rayon (93 percent) and spandex (7 percent) fibers; modal (94 percent) and spandex (6 percent) fibers; cotton and spandex fibers; and bamboo and spandex fibers. Materials that have stretch capability of equal to or greater than about 2× may be desired. Suitable examples of materials may have basis weights of about 0.09-0.15 gram/in.² per layer, or other basis weights.

Durable chassis materials may be selected to impart desired comfort, appearance and performance to chassis 20. In some circumstances it may be desired to select durable chassis materials which are sufficiently inexpensive to allow for disposal, if soiled extensively or damaged, with minimized issues of cost or conscience.

Semi-durable chassis materials may include any natural or synthetic nonwoven web and/or film materials known in the diaper or pant arts. Semi-durable materials of which chassis 20 may be constructed may include non-woven web materials of polypropylene and/or polyethylene fibers, polyester fibers, and any other synthetic fibers used to form nonwoven web materials used as components of disposable diapers, and blends thereof. Natural fibers such as cotton, linen, wool, bamboo, hemp, silk, rayon, and the like may be blended with synthetic fibers to form such a nonwoven web suitable as a component layer of chassis 20.

Non-limiting examples of fibers, nonwovens and laminates of nonwovens and films that might be considered for use as semi-durable chassis materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587.

Semi-durable chassis materials also may be selected to impart desired comfort, appearance and performance to chassis 20. In some circumstances it also may be desired to select semi-durable chassis materials which are sufficiently inexpensive to allow for disposal, if soiled extensively or damaged, with minimized issues of cost or conscience.

The chassis also, or additionally, may include a laminated or substantially separate film layer, which may be elastic, to provide enhanced liquid penetration resistance and/or elastic properties. Elastic properties also can be added or enhanced via the addition of other materials to the chassis in layer, band or strip fashion, including elastic strands, bands, scrims, and the like. A film layer may be laminated with a durable material or semi-durable material. A film layer may include an elastomer based on KRATON (a product of Kraton Polymers U.S., LLC, Houston, Tex.), or by way of further example, VISTAMAXX available from ExxonMobil Chemical Company, Houston, Tex.; FLEXAIRE, EXTRAFLEX or FABRIFLEX (products of Tredegar Film Products Corporation, Richmond, Va.), and various latex-free elastomeric sheets available from Fulflex Elastomerics Worldwide (Greenville, Tenn.).

Inclusion of an elastomeric material, either as a fibrous component of a cloth or nonwoven layer, or as a film layer, provides for improved stretchability and elasticity where it may be deemed useful to accommodate the wearer's anatomy and movements, such as over the wearer's buttocks and/or around the waist areas, and improved fit and comfort. Additionally, where a film layer may be included, it may impart additional liquid, containment capability to the chassis. A film layer may include a film that is substantially liquid impermeable, but vapor permeable, so as to provide breathability and reduce humidity within the chassis while it is being worn, reducing chances for overhydration of the skin where liquid containment capability is desired.

Referring to FIG. 2A, in one example outer surface 24 may be formed by a first layer of a durable or semi-durable material. The material selected may include fibers having hydrophobic properties, providing enhanced liquid containment attributes to such first layer. In another example, however, it may be desirable in some circumstances for the selected, material to include hydrophilic fibers, or fibers treated to be hydrophilic, so as will cause the material to more readily absorb and/or transmit liquid therethrough. This may serve to provide supplemental absorbency within the chassis for the event in which liquid exudates escape the insert, or to provide one way of communicating to the user that liquid exudates have escaped the insert. Additionally, in some circumstances it may be desirable that the material selected have soft tactile properties so as to have a pleasant feel that the user and/or wearer find attractive. The material also may be selected, so as to have a desired appearance, including but not limited to coloration, sheen, texture, etc.

Referring to FIG. 2B, in another example inner surfaces 25 may be formed by a second layer of a durable or semi-durable material. The material selected may include fibers having hydrophobic properties, providing enhanced liquid containment attributes to the second layer. In another example, however, it may be desirable in some circumstances for the selected material to include hydrophilic fibers, or fibers treated to be hydrophilic, so as will cause the material to more readily absorb and/or transmit liquid therethrough. This may serve to provide supplemental absorbency within the chassis for an event in which liquid exudates escape the insert, to provide one way of communicating to the user that liquid exudates have escaped the insert and/or to provide a layer that tends to draw moisture away from the skin, for a drier, more comfortable feel. Additionally, in some circumstances it may be desirable that the material selected have soft tactile properties so as to have a pleasant feel against the skin, particularly in areas where no portion of an insert is expected to be present between the chassis and the wearer's skin. In another example, the second layer of material may be formed of a textile material having enhanced elasticity, such as by inclusion of fibers of an elastomeric material (such as spandex). In another example, an intermediate film layer may be included, laminated or not laminated with another layer.

Forming the chassis 20 of more than one layer, for example, two, or more, layers, as described above, may provide various benefits. A second layer (and any additional layers) may provide supplemental tensile strength in both the lateral and longitudinal directions across the chassis 20. Additionally, a first layer may be selected for a first set of properties, and a second layer may be selected for a second set of properties. For example, material forming a first layer may be selected for having comparatively greater elasticity and a particular texture, color and/or other appearance-related properties, and material forming a second layer may be selected for having comparatively greater hydrophobicity and/or softness to the skin for purposes of an inner layer, the two layers in combination imparting a combination of desirable attributes to the chassis. Additionally, a plurality of layers may better serve to conceal bumps, corners, seams or other features of an insert, as compared with a single layer, for a smoother, more attractive appearance.

In addition to forming differing layers of differing materials, it may be desirable to form a single layer of differing materials, for example, differing materials in the respective front, crotch and/or rear regions of the chassis. Such differing materials may be joined at a seam such as inner seam 40 and/or outer seam 41. For example, the material predominately forming the inner surface of rear region 28 may be selected primarily for its elasticity features, which may better serve to provide snug fit about wearer body contours and accommodate wearer movement (i.e., about the buttocks and hips). By comparison, the material predominately forming the inner surface of front region 27 and/or crotch region 26 might be selected primarily for its hydrophobicity features, which may better serve to contain liquid exudates.

Layers or other elements of the chassis may be joined to each other via any suitable mechanism, including, for example, adhesives, mechanical bonding, ultrasonic bonding, sewing, stitching, serging, edging, and the like.

Elasticized Waistbands, Leg Bands

Referring again to FIGS. 1, 2A and 2B, front waist band portion 34, rear waist band portion 35, and leg band portions 36 are depicted. One or more of these band portions 34, 35, 36 may be formed of one or more strands or strips of an elastomeric polymer material such as spandex, enveloped by a nonwoven or textile material, which may include the edges of the material forming the inner and/or outer layers of chassis 20, to form and elasticize the respective band portions. The elastic material may be affixed. to or within a chassis layer in a strained. condition, or at zero applied strain. Textile material(s) enveloping the elastic strand(s) or strip(s) may be sewn around elastic strand(s) or strip(s) to hold them in place within the respective band portions. If the elastic material is strained prior to, and while, being enveloped and affixed to form these band portions during the manufacturing process, upon relaxation the enveloping material and adjacent chassis material may be caused to gather and form ruffles 37 therealong, which constitute gathered chassis material. This can serve to promote snug fit, wearer comfort and appearance. The band portion may be disposed along the edge of the chassis, and in some circumstances it may be desired to have the band portion situated along substantially the entire length of the leg and/or waist openings so as to form bands that substantially or completely encircle the wearer's legs and/or waist while chassis 20 is worn. The gathered material within ruffles 37 can serve to accommodate linear stretching of waist band portions 34, 35 and leg band portions 36. This arrangement including elasticized leg band portions 36 as described, not only may provide for better fit about the wearer's legs, but also may enable the chassis 20, when formed of appropriately sized and shaped material, to thrill a pouch-like structure 75 in the crotch region (see FIG. 1) when worn, which may serve to provide space within the chassis to accommodate the insert 50 (FIG. 3) and help hold it in place within chassis 20, in a substantially laterally centered position within the crotch region. This may be deemed advantageous in examples in which an insert 50 is attached within chassis 20 by fastener components only located proximate to the respective ends of insert 50, and not at any longitudinally intermediate locations, as described further below. Additionally, the snug fit provided by such elasticized leg band portions 36 may serve to enhance containment capability of the wearable absorbent article.

One or more of waist band portions 34, 35 and leg band portions 36 may be elasticized in the manner described above, or by other mechanisms. For example, elasticized band/strip material such as that used to form elastic waistbands and legbands of conventional cloth underwear and briefs may be separately produced, and affixed to the materials forming chassis 20 in any suitable manner, during the manufacture thereof.

In another example, one or more of waist band portions 34, 35 and leg band portions 36 may be formed of elastic material simply affixed about the leg opening and/or waist opening edges by use of adhesive and/or compression bonding In another example, an elastic strip material may formed by affixing a plurality of strained elastomeric strands or strips to one or more strips of unstrained nonwoven web material, or film. When the resulting elastic strip material is allowed to relax, the unstrained material forms transverse rugosities that comprise gathered unstrained material, which will accommodate stretching of the elastic strip material. By affixing the elastic strip material thereabout, the elastic strip material may be used to form one or more of waist band portions 34, 35 and/or leg band portions 36.

Anchoring Bands

Chassis 20 also may include an anchoring supplement such as anchoring band 38 disposed on or in the chassis rear region 28 as indicated in FIGS. 2A, 2B. As suggested in FIGS. 2A and 2B, anchoring band 38 may be affixed along a layer, or disposed between layers, forming inner surfaces 25 and outer surfaces 21 of chassis 20. Anchoring band 38 may include an elastomeric or elasticized strip or band of material, affixed to chassis 20 at locations proximate to its rearward corners or proximate to fastening ears 29. Thus, anchoring band 38 may he partially or substantially force-decoupled along its lateral length from the layer(s) forming the inner and outer surfaces of chassis 20, via attachment to the chassis only by the ends of anchoring band 38, or only at a limited number of selected intermediate lateral locations along anchoring band 38. For example, anchoring band 38 might be attached to chassis 20 only at the ends of anchoring band 38. In another example, anchoring band 38 might be attached to chassis 20 only at the ends and at the lateral center of anchoring band 38. This substantially force-decoupled arrangement allows anchoring band 38 and surrounding portions of chassis 20 to stretch and move substantially independently of one another, which may promote better fit and comfort. In another example, however, anchoring band 38 may be an elastic band, strip or strap laminated with or otherwise affixed to a layer of stretchable material forming either of or both the inner and outer surfaces of the chassis, along substantially the entire length of anchoring band 38.

When strained laterally by application to the wearer, anchoring band 38 may serve to provide, or supplement, lateral tensile forces in the article about the wearer's waist, thereby tending to draw the waist opening snug, enhancing fit and enhancing securement of the wearable absorbent article about the wearer's waist. The elastic modulus of the anchoring band may be higher than the elastic modulus of the surrounding, adjacent, or coextensive chassis materials.

An anchoring band, or system of one or more anchoring band members, may have any additional features described in, for example, co-pending U.S. patent applications Ser. Nos. 11/810,741; 11/810,708; 12/101,476; 12/028,317; 11/810,745; 11/810,742; 11/810,734; 11/810,779; 11/810,715; 11/810,733; 11/810,736, 11/810,777; and 11/599,862; 11/810,901 and 11/599,851; 11/899,812; 12/204,844; 12/204,849; 12/204,854; 12/204,858; and 12/204,864; 11/899,810; 11/899,656; and 11/899,811; 11/899,812; 12/204,844; 12/204,849; 12/204,854; 12/204,858; and 12/204,864.

In another example, instead of, or in addition to, being oriented substantially laterally as suggested by the depicted location of anchoring band 38 in FIGS. 2A and 2B, one or more members forming anchoring bands may be oriented diagonally between the longitudinal and lateral directions. For example, as suggested in FIG. 2A, a pair of diagonal anchoring bands 38a may have respective waist ends thereof affixed at a location area proximate to corners of the chassis and/or fastening ears 29, and respectively extend toward both the lateral and longitudinal center of chassis 20, as suggested in FIG. 2A. The respective center ends of bands 38a may be affixed to the chassis at locations proximate the lateral center of the chassis as suggested in FIG. 2A, and bands 38a may he either force-decoupled or force-coupled to the chassis along the lengths of bands 38a, as described above. In an example wherein an insert is connected to an anchoring band for additional longitudinal support as described further below, diagonal anchor bands such as diagonal anchor bands 38a may serve to provide supplementary longitudinal tension along chassis 29, providing supplemental longitudinal support therewithin.

### Examples of Possible Absorbent Insert Details

Examples of features of an absorbent insert 50 will be described with reference to FIGS. 3, 4 and 5A-F.

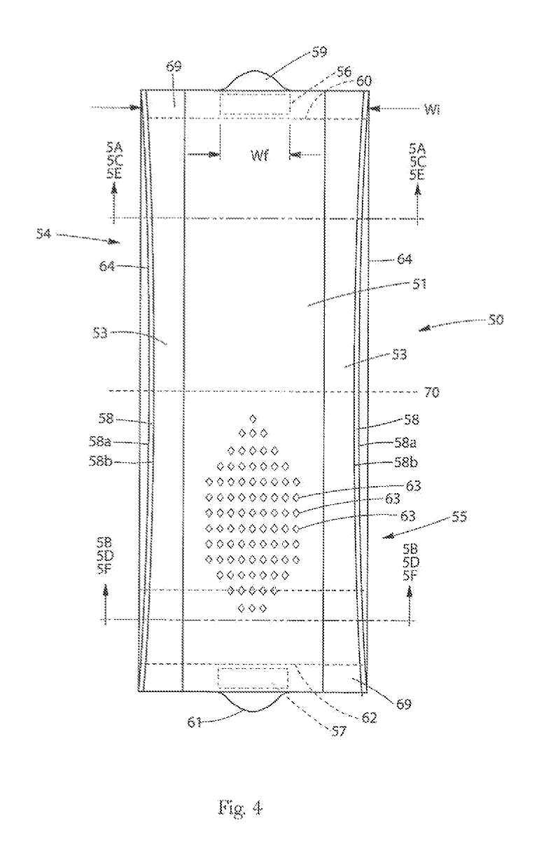

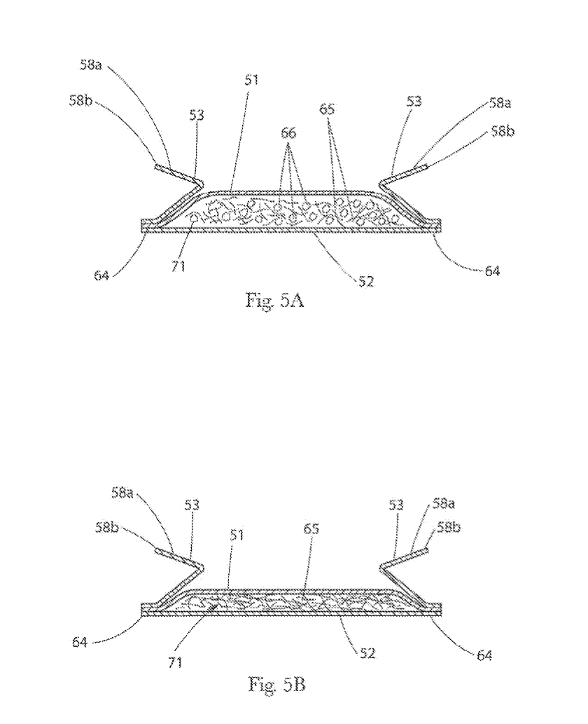

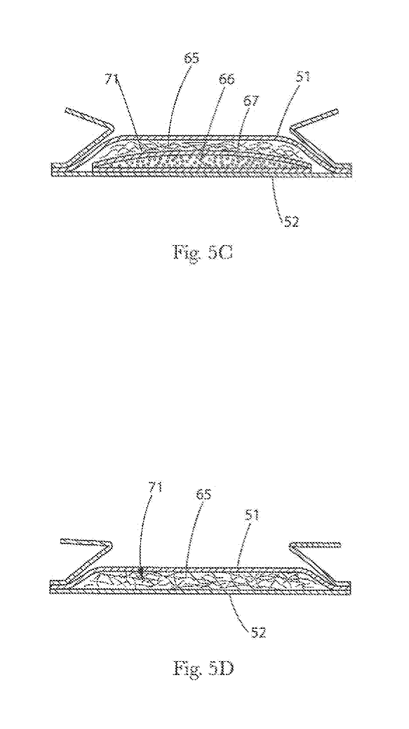

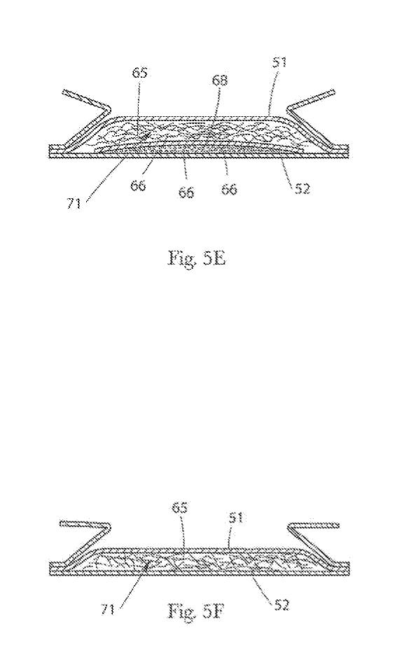

As noted above, FIG. 3 depicts a disposable absorbent insert 50 that may form an inner component of a wearable absorbent article as described herein, shown in perspective view as it might appear in a free-standing, relaxed state, apart from a chassis. FIG. 4 depicts an example of an insert 50 shown stretched out and laid flat (against elastic-induced contraction to a position similar to that shown in FIG. 3), body-facing surfaces facing the viewer. FIGS. 5A-5F depict cross sections of an insert 50 as indicated in FIG. 4, in various possible examples.

Insert 50 may have a topsheet 51 and backsheet 52 forming an envelope-like enclosure for absorbent core materials such as those described further below. Topsheet 51 and backsheet 52 may be affixed together along longitudinal seams 64, and along lateral seams 69, Insert 50 also may have longitudinal standing cuffs 53 affixed therealong.

Topsheet

Topsheet 51 may be formed of a liquid-permeable nonwoven web material. It may be desired that material forming topsheet 51 is compliant, soft-feeling, and non-irritating to the wearer's skin. It may be desired that at least a portion of topsheet 51 may be liquid pervious, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers (e.g., polyester or polypropylene fibers), or a combination of natural and synthetic fibers. If topsheet 51 includes fibers, the fibers may be spunbond, carded, wet-laid, meltblown, hydroentangled, or otherwise processed as is known in the art.

One suitable material comprising a nonwoven web of staple-length polypropylene fibers is manufactured by Veratec, Inc., a Division of International Paper Company, of Walpole, Mass. under the designation P-8, Additional suitable materials comprising formed films are described in U.S. Pat. Nos. 3,929,135; 4,324,246; 4,342,314; 4,463,045; and 5,006,394. Other suitable topsheets 30 may be made in accordance with U.S. Pat. Nos. 4,609,518 and 4,629,643. Suitable examples of formed and/or apertured films may include products produced by The Procter & Gamble Company of Cincinnati, Ohio as DRI-WEAVE, and by Tredegar Corporation, based in Richmond, Va., as FRESHFEEL. Suitable topsheet materials also may include laminates of films and nonwoven webs produced by Tredegar as COMFORTFEEL, COMFORTQUILT, SOFTQUILT and COMFORTAIRE.

In some circumstances it may be desired that at least a portion of topsheet 51 is made of a hydrophobic material or is treated to be hydrophobic in order to isolate the wearer's skin from liquids contained in absorbent core 71. If topsheet 51 is generally made of a hydrophobic material, it may be desired that at least a portion of the upper surface of topsheet 51 is treated to be hydrophilic so that liquids will transfer through the topsheet more rapidly. Topsheet 51 can be rendered hydrophilic by treating it with a surfactant or by incorporating a surfactant into the topsheet. Suitable methods for treating topsheet 51 with a surfactant include spraying the topsheet material with the surfactant and/or immersing the material into the surfactant. A more detailed discussion of such a treatment and hydrophilicity is contained in U.S. Pat. Nos. 4,988,344 and 4,988,345. A more detailed discussion of some suitable methods for incorporating a surfactant in the topsheet can be found in U.S. Statutory Invention Registration No. H1670. In another example, however, topsheet 51 may include an apertured web or film which is hydrophobic. This may be accomplished by foregoing the hydrophilizing treatment step from the production process and/or applying a hydrophobic treatment to the topsheet material, such as a polytetraflouroethylene compound like SCOTCHGUARD or a hydrophobic lotion composition, as described below. In such examples, it may be desired that the apertures be large enough to allow the penetration of aqueous fluids like urine without significant resistance otherwise attributable to hydrophobicity.

Any portion of topsheet 51 may be coated with a lotion as is known in the art. Examples of suitable lotions include those described in U.S. Pat. Nos. 5,607,760; 5,609,587; 5,635,191; 5,643,588; 5,968,025 and 6,716,441. The lotion may function alone or in combination with another agent as the hydrophobizing treatment described above.

Topsheet 51 may also include or be treated with antibacterial agents, some examples of which are disclosed in U.S. application Ser. No. 08/212,441, published as U.S. Statutory Invention Registration H1732.

Topsheet 51, backsheet 52 or any portion of the topsheet or backs et may be embossed and/or matte finished to provide a more cloth-like appearance.

Topsheet 51 may be folly or partially elasticized or may be foreshortened so as to provide avoid space between topsheet 51 and core 71. Exemplary structures including elasticized or foreshortened topsheets are described in more detail in U.S. Pat. Nos. 4,892,536; 4,990,147; 5,037,416; and 5,269,775.

Backsheet

Backsheet 52 is generally that outer liner portion of insert 50 forming the garment-facing surface thereof, and prevents the exudates absorbed. and contained within insert 50 from wicking through and soiling the chassis. In sonic circumstances it may be desired that backsheet 52 is substantially impervious to liquids.

Backsheet 52 may be formed of a film, a nonwoven, or a laminate of a film and a nonwoven. Backsheet 52 may be formed of a substantially liquid-impermeable laminate or composite of film and non-woven web. Backsheet 52 may be formed of a substantially liquid impermeable nonwoven web, or laminate of nonwoven web and substantially liquid impermeable film, so as to contain and isolate liquid exudates from the chassis, outer clothing and/or environment of the wearer. At the same time, backsheet 52 may be vapor permeable to provide for breathability of the insert and the wearable absorbent article, reducing humidity in the areas between the insert and the wearer's body, and helping reduce the likelihood of skin irritation and/or rashes that may result from over-hydration of the skin.

The material forming backsheet 52 may include a thin plastic film such as a thermoplastic film having a thickness of about 0.012 mm (0.5 mil) to about 0.051 mm (2.0 mils). Suitable backsheet materials also may be breathable materials which permit vapors to escape while still preventing liquid from passing therethrough. Suitable examples may include those manufactured by Tredegar Corporation, based in Richmond, Va., and sold under the trade names CPC2, X15306, X10962 and X10964 film. Other examples may include microporous films such as manufactured by Mitsui Toatsu Co., of Japan under the designation ESPOIR NO and by Exxon Chemical Co., of Bay City, Tex., under the designation EXXAIRE, and monolithic films such as manufactured by Clopay Corporation, Cincinnati, Ohio under the name HYTREL blend P18-3097. Some additional examples may include breathable composite materials as described in PCT Application No. WO 95/16746; and U.S. Pat. Nos. 5,938,648; 5,865,823; and 5,571,096. In other examples, backsheet 52 may comprise elastomeric films, foams, strands, or combinations of these or other suitable materials with nonwovens or synthetic films.

Backsheet 52 may be joined to topsheet 51, absorbent core 71 or any other element of insert 50 by any suitable attachment mechanism known in the art. For example, the attachment mechanism may include a continuous line or layer of adhesive, a patterned layer of adhesive, or an array of separate lines, spirals, or spots of adhesive. One example of an attachment mechanism comprises an open pattern network of filaments of adhesive as disclosed in U.S. Pat. No. 4,573,986. Other suitable attachment mechanisms include several lines of adhesive filaments which are swirled into a spiral pattern, as is illustrated by the apparatus and methods shown in U.S. Pat. Nos. 3,911,173; 4,785.996; and 4,842,666. Adhesives which have been found to be satisfactory are manufactured by H. B. Fuller Company of St. Paul, Minn. and marketed as HL-1620 and HL-1358-XZP. Alternatively, the attachment mechanism may comprise heat bonds, pressure bonds, ultrasonic bonds, dynamic mechanical bonds, or any other suitable attachment mechanisms or combinations of these attachment mechanisms known in the art.

It will be appreciated that the chassis described above can be constructed of materials and construction so as to bear and sustain a majority of the structural loading generally imposed upon a disposable diaper, through stretching and accommodation of the wearer's anatomical features and body movements, and through absorption, swelling and added weight resulting from the wearer's exudations of waste. Thus, lesser requirements for structural strength of an insert might be present with use of such a chassis, as compared with strength required of inside components of a disposable diaper. Therefore, an article such as described herein may include a disposable absorbent insert manufactured from materials that are different from those ordinarily used in the manufacture of disposable diapers, such as petroleum-derived materials, e.g., polyethylene and polypropylene. For example, a disposable absorbent insert having one or more of a topsheet, backsheet, standing cuffs and/or other components formed of wood and/or other cellulose fiber-based products (e.g., paper), in addition to the materials identified above, is contemplated. If resistance to aqueous liquid penetration or substantial liquid impermeability is desired, e.g., for a backsheet and/or standing cuffs, a material formed of ordinarily hydrophilic fibers such as paper may be coated. or impregnated with a hydrophobic material, such as a skin-compatible oil or wax, to impart the desired resistance to aqueous liquid penetration.

Absorbent Core

Referring to FIGS. 5A-F, insert 50 may have an absorbent core 71 within the envelope-like structure formed by topsheet 51 and backsheet 52. Absorbent core 71 may comprise any absorbent material which is generally compressible, conformable, non-irritating to the wearer's skin, and capable of absorbing and retaining liquids such as urine and other certain body exudates. Absorbent core 71 may comprise a wide. variety of liquid-absorbent materials commonly used in disposable diapers and other absorbent articles such as comminuted wood pulp, which is generally referred to as airfelt. Examples of other suitable absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

Absorbent core 71 may include liquid acquisition/distribution material 65, and storage material 66. Generally, acquisition/distribution material 65 may have comparatively rapid absorption and wicking properties, but also may have limited absorption capacity. Conversely, generally, storage material 66 may have comparatively slower absorption and wicking properties, but also may have greater absorption capacity. Thus, acquisition/distribution material 65 may serve to rapidly absorb and distribute gushes of liquid such as urine, while storage material 66, having greater absorption capacity, may serve to absorb such liquid from the acquisition/distribution material and store it for the time needed until the insert may be replaced.

Absorbent core 71 may be manufactured in a wide variety of sizes and shapes (e.g., rectangular, hourglass, “T”-shaped, etc.). The configuration and construction of absorbent core 71 may also be varied (e.g., the absorbent core(s) or other absorbent structure(s) may have varying caliper zones, hydrophilic gradient(s), a superabsorbent gradient(s), or lower average density and. lower average basis weight acquisition zones; or may comprise one or more layers or structures). Examples of absorbent structures for use as absorbent core 71 may include those described in U.S. Pat. Nos. 4,610,678; 4,673,402; 4,834,735; 4,888,231; 5,137,537; 5,147,345; 5,342,338; 5,260,345; 5,387,207; and 5,625,222.

To reduce the overall size and/or thickness of the absorbent core, and thereby improve wearer comfort and reduce the volume of disposable waste created by a soiled insert, it may be desired to construct an absorbent core using the lowest volumes of core materials possible within performance constraints. Toward this end, examples of suitable materials and constructions for a suitable absorbent core are described in, but are not limited to, copending U.S. application Ser. No. 12/141,122.

Sublayer

Insert 50 may also include a sublayer disposed between topsheet 51 and backsheet 52, The sublayer may be any material or structure capable of accepting, storing or immobilizing bodily exudates. Thus, the sublayer may include a single material or a number of materials operatively associated with each other. Further, the sublayer may be integral with another element of insert 50 or may be one or more separate elements joined directly or indirectly with one or more elements of insert 50. Further, the sublayer may include a structure that is separate from the core 71 or may include or be part of at least a portion of the core 71.

Suitable materials for use as the sublayer may include large cell open foams, macro-porous compression resistant nonwoven highlofts, large size particulate forms of open and closed cell foams (macro and/or microporous), highloft nonwovens, polyolefin, polystyrene, polyurethane foams or particles, structures comprising a multiplicity of vertically oriented looped strands of fibers, absorbent core structures described above having punched holes or depressions, and the like. One example of a sublayer includes a mechanical fastening loop landing element, having an uncompressed thickness of about 1.5 millimeters available as XPL-7124 from the 3M Corporation of Minneapolis, Minn. Another example includes a 6 denier, crimped and resin-bonded nonwoven highloft having a basis weight of 110 grams per square meter and an uncompressed thickness of 7.9 millimeters which is available from the Glit Company of Wrens, Ga. Other suitable absorbent and nonabsorbent sublayers are described in U.S. Pat. Nos. 6,680,422 and 5,941,864. Further, the sublayer, or any portion thereof, may include or be coated with a lotion or other known substances to add, enhance or change the performance or other characteristics of the element.

Standing Cuffs

Insert 50 also may have a pair of longitudinal standing cuffs 53 attached partially or entirely along the length thereof. Suitable longitudinal standing cuffs (in various published. examples identified as “leg cuffs”, “barrier cuffs” “gasketing cuffs,” etc., may be formed of materials and construction such as described in, but not limited to,

U.S. Pat. Nos. 5,769,838 and 4,597,760; and copending U.S. Published Application No. 2007/0239130. As shown in FIG. 3, standing cuffs 53 may have one or more strands or strips of cuff elastics 58a, 58b disposed longitudinally therealong. If such cuff elastics 58a, 58b are pre-strained prior to being affixed to the web material forming standing cuffs 53, resulting longitudinal tensile forces therealong will cause the web material forming standing cuffs 53 to gather as shown, and cause the cuffs to extend from the body of the insert (upwardly relative to FIG. 3), or causing them to “stand”. This feature causes standing cuffs 53 to form a gasketing structure along the wearer's body when the article including insert 50 is worn, longitudinally on either side of the anatomical features where waste is exuded. Thus, standing cuffs 53 may serve to enhance the exudate containment capability of insert 50 and, and as a result, of the wearable absorbent article. As with backsheet 52, standing cuffs 53 may be formed of a substantially liquid impermeable web so as to contain and isolate liquid exudates from the chassis, outer clothing and environment of the wearer. At the same time, standing cuffs 53 may be vapor permeable to provide for breathability of the insert and the wearable absorbent article, reducing humidity in the areas between the insert and the wearer's body, and helping reduce the likelihood of skin irritation and/or rashes that may result from over-hydration of the skin.

In another example, the material forming standing cuffs 53 may be integral with the material forming backsheet 52, such as described in, by way of non-limiting example, copending U.S. Published App. No. 2007/0239130. In this particular example, referring to and relative to the view in FIG. 3, a continuous piece of material may form one standing cuff 53, wrap beneath the insert to form backsheet 52, and wrap up the other side to form the other standing cuff 53. This example of a wraparound construction may provide improved liquid containment capability to insert 50, by eliminating seams along the outer liquid-containing surfaces that include standing cuffs 53 and backsheet 52. In some circumstances, however, manufacturing and/or economic constraints may discourage such construction, or else, it may be desirable for the materials forming standing cuffs 53 and backsheet 52 to have differing properties. For example, in some applications it may not be deemed necessary for standing cuffs 53 to be substantially liquid impervious, if they are otherwise formed of a nonwoven web material comprising closely situated hydrophobic fibers, which may still tend to repel and contain fluid, but may be generally more breathable than substantially liquid impervious laminates including films, in this event, improved strength and liquid containment attributes can still be imparted by having the material forming standing cuffs 53 wrap only partially beneath the lower longitudinal corners of the insert, and affixed at seams beneath the insert, rather than at its outermost lower corners as suggested by FIG. 3.

Grasp Structures

Referring to FIGS. 3 and 4, insert 50 also may include respective forward and rearward user grasp structures 59, 61. User grasp structures 59, 61 may be provided to enable the user to quickly and easily grasp insert 50 proximate its respective ends.

Grasp structures as shown and/or suggested may enable the user to more quickly grasp and stretch insert 50 from a contracted position similar to that depicted in FIG. 3, to an extended position similar to that depicted in FIG. 4, which may be desirable for installing insert 50 into a chassis. If user grasp structures 59, 61 are centered proximate to the respective ends of insert 50 as shown, this may also provide visual assistance to the user for co-locating respective centered fastener component pairs, described in more detail below.

Additionally, user grasp structures 59, 61 may serve to enable the user to quickly and easily grasp insert 50 proximate to its respective ends, which as a result of their distance from exudation points on a wearer's body, are less likely to be soiled at the time replacement of insert 50 becomes necessary or desirable. Thus, the user may be better enabled to avoid contacting the wearer's exudates with the user's hands when removing a soiled insert 50 from a chassis 20.

User grasp structures 59, 61 may include tab-like extensions as shown in FIGS. 3 and 4, with free ends unattached to the chassis 20 when insert 50 is installed therein, which are easily graspable. User grasp structures may have different forms as well. By way of non-limiting example, user grasp structures may take the form of loop-like extensions (not shown) extending from the ends of insert 50, finger holes (not shown) through insert 50 proximate the ends thereof, and other structures that facilitate grasping and pulling of insert 50 at locations proximate to its ends.

### Insert/Chassis Fastener Components; Orientation Indicia; Other Possible Features

Referring back to FIGS. 2B, 3 and 4, as previously noted, chassis 20 may have one or more insert fastener components such as front and/or rear insert fastener components 33, disposed thereon. Insert 50 may have front and/or rear fastener components 56, 57 disposed thereon. Respective front and/or rear fastener components 56, 57 on insert 50 may be selected and/or adapted to be cooperative to enable fastening with respective front and/or rear insert fastener components 33, 32 disposed on chassis 20.

Types, Locations and Localization of Fastening Locations

In one example, to enable fastening of respective front and rear fastener components 56, 57 of insert 50 with respective front and rear insert fastener components 33, 32 on chassis 20, respective fastening pairs 56, 33 and 57, 32 may include cooperating fastener components. An example of a suitable hook-and-loop fastening system is a VELCRO system, a product of Velcro Industries B.V., components of which are available from Velcro USA, Inc., Manchester, N.H. A hook-and-loop fastening system provides certain advantages. Because the respective hook and loop components are supplied in sheet or strip form, they may be cut into suitably shaped patches that can be affixed to a cloth substrate by various mechanisms, including adhesive bonding, mechanical bonding, ultrasonic bonding, sewing, stitching, serging, edging, and the like. If respective hook-and-loop patches are used as fastener components, relative ease of fastening, simplicity and convenience for the user (as compared with, for example, fastener components such as a button and button hole) are one among several advantages provided, because fastening is effected simply by placing the fastener components in face-to-face contact and applying gentle pressure.

Some types of hook components may, in some circumstances, tend to snag or catch undesirably on a variety of materials in addition to intended corresponding loop components, while most types of loop components currently available do not have this tendency. Thus, in some circumstances it may be desired that patches of loop components form one or both of insert fastening elements 33, 32, while patches of hook components form one or both of fastening elements 56, 57. This arrangement places a non-snagging insert fastening component on the chassis. This may be desirable in some circumstances, such as when the chassis 20 is designed to be reusable—reducing the likelihood that components on a chassis 20 will undesirably snag on other parts of chassis 20 or on other articles, such as clothing articles, being stored or laundered along with chassis 20.

However, fastening pairs 56, 33 and 57, 32 need not necessarily include respective components of a hook-and-loop fastening system, and need not necessarily include respective components of a two-component fastening system. Rather, a fastening system may require only one fastening component, or use other types of fastener components. Fastener components used may be adapted to engage, retain, and otherwise hold the insert or a portion thereof. A fastener component on chassis 20 may include a patch of adhesive; a structure having a region of relatively high coefficient of friction; a pocket; flap; strap; or other capturing, holding and/or retaining surface, device or structure. Thus, in one example, the inside of chassis 20 may include a pocket structure situated on or along the inner surface 25 of chassis 20, in, e.g., the front region 27. Such a pocket structure may have an opening facing downward or upward (relative to the wearer in a standing position, and relative to FIG. 2B). The pocket structure may be adapted to receive, fit and capture, for example, the forward edge and a portion of forward region 54 of insert 50. Such a fastener component may be provided in lieu of, or in addition to, other fastener components disposed at the front region of the chassis 20 and forward region of insert 50. With such a structure and a suitably adapted insert, to install the insert the user may insert the forward edge of insert 50 into the pocket structure, and then fasten the rearward portion of insert 50 into the rear region of the chassis 20 by any other rear fastener component(s) provided.

Also, a fastener component in any form, including a respective component of a fastening pair 56, 33 and 57, 32 may be disposed on either of insert 50 or chassis 20. For example, a hook patch may be disposed on either of insert 50 or chassis 20, with a cooperating loop patch disposed on the other of insert 50 or chassis 20.

An insert fastener component such as component 32 on chassis 20 may be attached or connected to an anchor band 38, or diagonal anchor band 38a. This may provide, as one advantage, the distribution of structural loading resulting from the weight of absorbed or contained exudates, as described in one or more of the patent applications cited above, in the description of anchor hands.

Fastening locations may be multiply disposed, for example, at each of the four corners of insert 50 and corresponding locations on chassis 20, and even at other locations along insert 50 and chassis 20, created by fastener components suitably selected and disposed.

In another example, however, a fastening system such as either of fastener component pairs 56, 33 and 57, 32 may form a singularized and localized fastening location proximate each of or either, the forward/front and/or rearward/rear ends/regions as suggested in the Figures. Having only two respectively singularized, localized fastening locations substantially laterally centered on the insert and the chassis, as suggested in FIGS. 2B and 3, provides an advantage of simplicity and ease of installation for the user, by presenting only two locations at which the user must co-locate and fasten insert 50 to chassis 20.

Additionally, having a single, localized fastening location proximate only one or two ends of insert 50, such as suggested in the Figures, provides a partially force-decoupled attachment of insert 50 within chassis 20. With this force-decoupled arrangement, the portions of chassis 20 surrounding fastener component(s) 33, 32, such as waistband portions 34, 35, and front and rear regions 27, 28, may laterally stretch and contract substantially independently of and unimpeded by the structure of insert 50 and portions thereof. This may avoid lateral bunching of the ends of insert 50 with lateral contraction of the chassis 20, and/or, conversely, may avoid having the structure of insert 50 restrict chassis 20 from stretching or contracting laterally, as a result of a more force-coupled. arrangement therebetween. Such lateral bunching or restricting otherwise may be incidental to fastening insert 50 within chassis 20 in a manner that force-couples a greater portion of the width of insert 50 to chassis 20, e.g. by use of more than one fastening location across the width of insert 50 proximate a given longitudinal location, or by use of a laterally delocalized/extended continuous fastening location along a larger portion of the width of insert 50, Force-decoupling, therefore, may provide for enhancement of fit, appearance and wearer comfort, as well as improved capability for maintenance of intended insert shape and performance while worn.

Thus, in one example, referring to FIG. 4, a patch of hook components forming a fastening component 56 attached to insert 50 may he substantially laterally centered on the insert as suggested in the figure, and may have an width W_{f} attached along insert 50 that extends no more than about 50 percent of the lateral width Wⁱ of the insert 50 at forward region 54. This provides that no more than about 50 percent of the width of the insert 50 at, e.g., forward region 54, will be force-coupled to the chassis by operation of the patch of hook components, and that, correspondingly, about 50 percent of such width (i.e., that portion of width Wᵢ that is not attached. to, or force-coupled to, the insert along width W_{f}) is force-decoupled from the chassis 20 when insert 50 is installed therein. In some circumstances, it may be desired that the portion of the lateral width of forward region 54 and/or of rearward region 55 of insert 50 that is force-decoupled from the chassis is even greater than about 50 percent. Thus, it may be desired that the width W_{f} of a fastening component 56 or 57, comprising, e.g., a continuously attached patch of hooks, is no more than about 40 percent, no more than about 30 percent, or even no more than about 20 percent, of the lateral width Wᵢ of the insert at the region where the fastening component is situated. In another example, it may be desired in some circumstances that at least about 50 percent of the lateral width of the insert 50 in the front and/or rear region 54, 55, is force-decoupled from the chassis. In some circumstances it may be desired that more than about 50 percent, more than about 60 percent, more than about 70 percent, or even more than about 80 percent, of the lateral width of the insert at any longitudinal location, is force-decoupled from the chassis when installed therein.

Other fastener components that provide such singularized and relatively laterally localized fastening locations, providing limited force-coupling across the lateral width of insert 50, are possible.

Insert Lateral Stiffeners

Referring to FIG. 3, and from the description of standing cuffs 53 with cuff elastics 58a, 58b set forth above, it can be appreciated that, if longitudinal tensile forces are imparted by pre-strained cuff elastics 58a. 58b, standing cuffs 53 may tend to pull the respective outer corners of insert 50 longitudinally toward each other. If there is not structure present to resist this pull, the outer corners of insert 50 may buckle and bunch, and standing cuffs 53 may collapse or otherwise be loose and less effective as gasketing structures against the wearer's body. In one example, one way of providing resistive structure is to either include insert-chassis fastener systems disposed at fastening locations at, or extending to, the corners of insert 50, and corresponding locations along the inner surfaces 25 of chassis 20. In some circumstances, however, this may be undesirable for reasons of cost and/or complexity. It also may sacrifice the advantages of having only one singularized/localized fastening location proximate each end of insert 50, as described above.

If only one singularized and relatively localized fastening location is provided at one or both ends of insert 50 as described, immediately above, and sized and located so as to be in a laterally centered position as suggested by the Figures, it may be desirable to include a lateral stiffening component proximate to one or both of such ends. Referring to FIGS. 3 and 4, lateral stiffeners 60 and 62 may be included. Such lateral stiffeners may serve to aid the user in engaging the insert with the chassis, and to help insert 50 maintain its intended shape and configuration while being worn beneath a chassis.

Lateral stiffeners 60, 62 may be affixed to, or incorporated within, insert 50 proximate the ends thereof as suggested by the Figures. In addition to increasing the tendency of insert 50 to maintain optimal shape while in use, such lateral stiffeners may increase the tendency of the respective ends of insert 50 to stay open and flat before insert 50 is installed in a chassis. Because the ends of insert 50 may otherwise be folded over or bunched while being stored and/or carried by the user before installation, lateral stiffeners may enhance user convenience, by cousin the ends of insert 50 to maintain or seek a shape/configuration that requires less manipulation by the user to install it in a chassis.

Referring to FIG. 4, one or more lateral stiffeners 60, 62 may be formed of any fiat, sheet-like or card-like material, or any flat, stiffened assembly. In one example, a lateral stiffener may be formed by folding over portion of an insert (such as an end portion) to create a stiffened. region comprising folded layers of material. In another example, lateral stiffeners 60, 62 may be formed by depositing onto the ends of insert 50 lateral bands or strips of liquid or semi-liquid adhesive or other material that cures or cools to a stiffened state, and thereby imparts added stiffness to the substrate to which it is applied. In another example, lateral stiffeners may be formed of cardboard or like material. One example of suitable stiffener material is 0.031 in. thick VOLARA foam supplied in sheet form (a product of Sekisui Voltek, LLC, Lawrence, Mass.). Stiffener materials may be laminated with or adhesively applied to portions of insert 50 to be stiffened, or applied, affixed or included by any other suitable method.

The lateral stiffeners may increase planar and/or lateral stiffness of the areas of insert 50 in which they are located, as compared with portions of the insert adjacent such stiffeners with respect to a plan view (such as FIG. 4). As a measure of relative stiffness of a stiffened portion (i.e., a portion including a lateral stiffener), compared with stiffness of an adjacent non-stiffened portion of an insert, Buckling Forces of each portion may he measured and compared according to the Buckling Force Test Method set forth in copending Published U.S. Application, Pub. No. 2007/0142798. The ratio of the Buckling Force of a portion of an insert having a lateral stiffener to the Buckling Force of an adjacent portion may be at least about 2.0, but in some circumstances it may be desired that the ratio be at least about 2.5, or even 4.0, or even 10.0, or more. It may be desired that the portions of insert 50 including one or more lateral stiffeners 60, 62 are sufficiently stiff to effectively resist the tension forces in standing cuffs 53 and substantially maintain standing cuffs 53 in their gasketing configurations while insert 50 is in use, and substantially maintain the corners of insert 50 in laterally extended positions, preventing buckling or bunching of the same. The amount of stiffness desired may depend upon various factors including the inherent stiffness of the insert materials without a supplemental lateral stiffener, and the amount and/or range of tension in the standing cuffs when the article is worn. At the same time, for purposes of wearer comfort and safety, it may be desirable that the portions of the insert including the one or more lateral stiffeners 60, 62 are pliable enough to flex comfortably with the wearer's body movements, and to collapse before any form of contusion or impalement injury hazard would be presented. It also may be desirable that stiffened portions are elastic in nature, in they will tend to return to a substantially flat configuration after being bent, folded or twisted.

Lateral stiffeners 60, 62 may be located adjacent or near one or both ends of insert 50 and may extend laterally from the lateral center thereof to stiffen the insert along a substantial portion of its width. The one or more lateral stiffeners may have a width of from about 50 percent to about 100 percent of the lateral width of the insert, or may extend beyond the longitudinal edges of other materials forming the insert. The lateral stiffeners may have any longitudinal dimension, although, in some circumstances, a longitudinal dimension less than 25 percent of the insert length may better assure comfort for the wearer, and therefore, may be desired. In some examples, the longitudinal dimension of a lateral stiffener may range from about 5 mm to about 50 mm. A stiffener also may extend longitudinally beyond the lateral edge of other materials forming the insert. One or both ends of the insert may include a lateral stiffener. In examples wherein the insert comprises more than one lateral stiffener, the respective lateral stiffeners may have differing shapes, dimensions, stiffness, thickness, color, structure, placement, material(s) or composition. A lateral stiffener also may include, or be integral with, a grasp structure as described above, and as suggested in FIG. 3 (lateral stiffener 60 is depicted as integral with grasp structure 59).

Targeting and Orientation Indicia

The insert and/or chassis may comprise one or more insert targeting indicia to indicate, facilitate and/or compel correct positioning and association of portions of the insert within the chassis. The insert targeting indicia may comprise verbal or non-verbal instructive indicia, visual targeting indicia, cooperating geometrical features, cooperating types of fastener components, or cooperating, designs of fastener components sized and formed to indicate or compel the engagement of the insert with the correct region of the chassis so as to enable the optimum performance of the wearable absorbent article. Other examples of possible targeting indicia components include one or more cooperating colors, shapes, patterns, lines, outlines, silhouettes, other geometrical features, protrusions or depressions, textures, patterns, targeting lines or crosshairs, bulls-eye representations, and the like, disposed on the chassis and/or the insert to indicate correct positioning of the insert within the chassis. In one example, the inner surface of the chassis may be imprinted with an outline of an insert, or a silhouette of an insert.

Targeting indicia also may comprise at least two cooperating components, one on the chassis 20 and one on insert 50, such that when these two components are associated, the respective components of chassis/insert system will be properly oriented with respect to one another and. will perform most optimally. In one example, the inner surface of the chassis may be imprinted with a first arrow pointing at an insert location, and the insert may be imprinted with a second arrow pointing at the first arrow when the insert and chassis are correctly relatively positioned.

Indicia may he cognitively correlating, or non-correlating, a correlation indicating a correct optimal placement, and a non-correlation indicating an incorrect sub-optimal placement. Respective cognitively correlating targeting indicia may include an indicium on the chassis that cognitively correlates with an indicium on the insert, indicating to the user the correct relative positioning and engagement of the insert and the chassis. For example, respective cognitively correlating indicia on the insert and chassis may have a common color, shape, or texture. (As used herein, “common color” includes any first color and recognizable shades or variants thereof, which in view of all features of the article is visibly and cognitively distinguishable from another color.)

Referring to FIGS. 2B, 3 and 4, when an insert 50 is asymmetrical as described above, it may have only one optimal forward region 54 and only one optimal rearward region 55. Similarly, when a chassis 20 is asymmetrical as described above, it may have only one optimal front region 27 and only one optimal rear region 28. Thus, in the event either or both of these asymmetries are substantial, installation of insert 50 into chassis 20 with incorrect relative front-rear orientation may cause the wearable absorbent article not to fit and/or function optimally. Accordingly, it may be desirable in some circumstances to incorporate one or more indicia into the chassis 20 and/or insert 50 that are adapted to inform the user as to the correct respective front-rear orientation of these components. Such indicia may provide such information to the user functionally, tactilely and/or visually.

Functional indicia may include fastener components that function properly, effectively and/or optimally with correct front-rear orientation, but do not function properly, effectively and/or optimally with incorrect front-rear orientation.

For example, referring to FIGS. 2B and 3, front insert fastener component 33 on chassis 20 may be cooperative to effect optimal/maximum fastening security only with front fastener component 56 on insert 50, but not with rear fastener component 57 on insert 50. Similarly, rear insert fastener component 32 on chassis 20 may be cooperative to effect optimal/maximum fastening security only with rear fastener component 57 on insert 50, but not with front fastener component 56 on insert 50.

In a more specific example, front insert fastener component 33 on chassis 20 may include a patch of loops, while front fastener component 56 on insert 50 may include a patch of hooks. Correspondingly, rear insert fastener component 32 on chassis 20 may include a patch of hooks, while rear fastener component 57 on insert 50 may include a patch of loops. Thus, in this particular example, if a user mistakenly attempts to fasten rear fastener component 57 on insert 50 (loops) to front insert fastener component 33 on chassis 20 (loops), proper or optimal fastening will not be effected, which will communicate to the user that he/she must rotate insert 50 by 180 degrees to install it with correct/optimal front-rear orientation on the chassis.

In another specific example, front insert fastener component 33 on chassis 20 may include a female snap fastener component, while front fastener component 56 on insert 50 may include a male snap fastener component. Correspondingly, rear insert fastener component 32 on chassis 20 may include a male snap fastener component, while rear fastener component 57 on insert 50 may include a female snap fastener component. Thus, in this particular example, if a user mistakenly attempts to fasten rear fastener component 57 on insert 50 (female snap fastener component) to front insert fastener component 33 on chassis 20 (female snap fastener component), the components will not fit properly together and proper/optimal fastening will not be effected, which will communicate to the user that he/she must rotate insert 50 by 180 degrees to install it with correct/optimal front-rear orientation.

Thus, functional indicia may include any fastener components that will function properly and/or optimally to effect fastening and maximum fastening security between insert 50 and chassis 20 when the two are properly oriented, but will not function properly or optimally otherwise. Additionally, functional indicia are not necessarily limited to fastener components. Functional indicia also may be embodied in other features of the chassis and insert that affect how the two fit or function together in correct, optimal front-rear orientation versus incorrect (reverse), sub-optimal front-rear orientation. Thus, functional indicia may additionally be associated with or combined with another functional element of the chassis or insert. Indicia may be associated with elements of the chassis such as a waistband, side panel, stretch element, leg cuff, physical retention fastener component (e.g., a pocket or retaining strap), and the like, Indicia may be associated with elements of the insert, such as a waist cap, waist band, standing cuff, fecal management feature, insert positioning aid, insert stiffening aid, insert removal aid, or insert disposal aid.

From the foregoing it will be appreciated that other forms of functional orientation indicia are possible, within the principle of the foregoing description.

In other possible examples, instead of respective functionally cooperative/uncooperative pairs of components as described above, insert 50 and chassis 20 may include respective non-functional indicia, such as tactile or other sensory indicia.

For example, the front insert and chassis fastener components may be selected or formed so as to have a first tactile attribute, while the rear insert and chassis components may be selected or formed so as to have a second tactile attribute. In another example, features of the insert and chassis may have features such as 3-dimensional shapes that are mating or geometrically cooperating with optimal front-rear orientation, but not mating or geometrically cooperating with reversed, suboptimal front-rear orientation.

In other possible examples, instead of or in addition to respective functionally cooperative/uncooperative pairs of fastener components or tactile indicia components as described above, insert 50 and chassis 20 may include respective visual indicia. Chassis 20 may include respective front and rear visual indicia 73, 74 disposed on the front region 27 and rear region 28, respectively. Respective visual indicia disposed on insert 50 and chassis 20 may be adapted to provide a visual cue to the user of correct/optimal orientation and placement of insert 50 within chassis 20.

For example, components of visual indicia and a visual cue may involve use of a common color. In one particular example, front and/or rear visual indicia 73, 74 disposed on chassis 20 may comprise respective common colors visibly distinct from one another. Insert 50 may have respective cooperating indicia disposed or embodied thereon. Thus, for example, forward and rearward user grasp structures 59, 61 on insert 50 may bear or be colored with colors respectively common and corresponding with those comprised by front and/or rear visual indicia 73, 74. More particularly, for example, front visual indicium 73 and an insert feature such as forward. user grasp structure 59 may both bear or be colored a first common color, and rear visual indicium 74 and a feature such as rearward user grasp structure 61 may either or both be colored a second common color, visibly distinguishable from the first common color.

In another particular example, front and rear visual indicia 73, 74 disposed on chassis 20 may embody, or bear images of, respective shapes to match, or cognitively correlate visually correlate with, corresponding shapes embodied, or pictured on, insert features such as forward and rearward user grasp structures 59, 61. More particularly, for example, front visual indicium 73 and forward user grasp structure 59 may both embody or bear images of circles, and rear visual indicium 74 and rearward user grasp structure 61 may both embody or bear images of triangles.

In another particular example, front and rear visual indicia 73, 74 disposed on chassis 20 may bear images of words or symbolic indications for “front” and “rear”, to match corresponding words or symbolic indications on insert features such as forward and rearward user grasp structures 59, 61. In a more particular example, front visual indicium 73 and forward user grasp structure 59 may both bear an image of the letter “F” (i.e., for “front”), and rear visual indicium 74 and rearward user grasp structure 61 may both bear an image of the letter “R” (i.e., for “rear”).

In another particular example, front and/or rear visual indicia 73, 74 disposed on chassis 20 may embody, or bear images of, respective portions of expectedly user-recognizable shapes, characters, objects, etc., to match corresponding portions of expectedly user-recognizable shapes, characters, objects, etc., embodied, or pictured on, insert features such as forward and/or rearward user grasp structures 59, 61. In a more particular example, front visual indicium 73 and forward user grasp structure 59 may both embody or bear respective portions of a first image that, when brought together properly, form a cognitively complete image of an expectedly user-recognizable first shape, character, object, etc.; and/or rear visual indicium 74 and rearward user grasp structure 61 may both embody or bear respective portions of a second image that, when brought together properly, form a cognitively complete image of an expectedly user-recognizable second shape, character, object, etc., distinctive from the first.

In yet another particular example, visual orientation indicia may be simplified into a single pair of visual indicia appearing, respectively, on chassis 20 and insert 50. In one such example, an inner surface 25 of chassis 20 may bear an image of an arrow pointing longitudinally toward front edge 21, and insert 50 may bear an image of an arrow pointing longitudinally toward its forward end.

Examples of other suitable visual orientation indicia adapted to provide orientation information may include alphanumeric text including words, arrows, symbols, diagrams, pictographs, icons, cartoons, schematics, and any other visual indicia.

It may be desired that indicia associated with the front portions of the chassis and insert will not cognitively correlate, or will cognitively not correlate, with indicia associated with rear portions of the chassis and insert, and vice versa, when the user views both sets of indicia.

From the foregoing it will be appreciated that other forms of visual orientation indicia are possible, within the principle of the foregoing description.

A two-piece wearable absorbent article having some or all of the features described herein may provide advantages over both conventional wholly reusable cloth diapers and conventional wholly disposable diapers. The potential for use of semi-durable materials, and more so durable materials, to form a chassis, provides for a chassis that may be used more than once, and, depending upon the materials selected, used and laundered many times. A chassis having some or all of the features described herein may eliminate the necessity for a disposable chassis structure, thereby reducing the volume of soiled waste the user must dispose of, as compared. with typical disposable diapers. Additionally, because the possibility of a reusable chassis that bears most of the structural loading generally imposed upon a disposable diaper is presented, disposable absorbent portions may have more simplified designs, reducing manufacturing and material costs as compared with those of disposable diapers. The possibility for making a disposable absorbent insert of non-traditional renewable materials (such as paper) is presented. At the same time, a disposable absorbent insert and chassis having some or all of the features described herein may in many circumstances prevent most or all soiling of the chassis by the wearer's exudates, thereby mitigating sanitation and odor problems associated with handling and storage, reducing the frequency of laundering necessary, and reducing the need for laundering resources, efforts and/or expenses, associated with conventional cloth diapers. A disposable absorbent insert having some or all of the features described herein also may provide better absorbency and better isolation of exudates from both the wearer's skin, and the wearer's clothing and environment, than conventional cloth diapers.

Use of durable materials for a chassis also may provide other incidental benefits, in creating choices in use of materials for improved and/or more appealing comfort, fit, designs, colors, patterns, etc. as compared with disposable diapers. A chassis having features described herein provides a wide variety of choices for making a wearable absorbent article look more attractive and/or more like an article of clothing or outerwear. In addition to the foregoing advantages, the use of an insert having an asymmetric structure together with orientation indicia allows for the design of an insert tailored to wearer anatomy and bodily functions as they differ front-to-rear, better performance, and increased, economy in design, construction and use of materials, while enabling the user to ensure correct front-rear orientation of the insert within the chassis. Other advantages are apparent from the description above.

The reusable chassis and the absorbent inserts disclosed herein, including any alternative embodiment, can be configured and put together in any way disclosed herein or known in the art.

In various embodiments, any of the reusable chassis, any of the absorbent inserts, and any of the resulting wearable absorbent articles disclosed herein can be configured as described in any of the following: published U.S. patent application 20100179496, entitled “Disposable Absorbent Insert for Two-Piece Wearable Absorbent Article”; published U.S. patent application 20100179498, entitled “Reusable Wearable Absorbent Articles with Anchoring Systems”; published U.S. patent application 20100179499, entitled “Reusable Outer Covers for Wearable Absorbent Articles”; published U.S. patent application 20100179500, entitled “Two-Piece Wearable Absorbent Article”; published U.S. patent application 20100179501, entitled “Outer Cover for Two-Piece Wearable Absorbent Article”; published U.S. patent application 2011017262 entitled “Article of Commerce Including Two-Piece Wearable Absorbent Article”; published U.S. patent application 20100179502, entitled “Reusable Wearable Absorbent Articles with Anchoring Systems”; copending U.S. patent application Ser. No. 12/785,152 entitled “Insert With Advantageous Fastener Configurations and End Stiffness Characteristics for Two-Piece Wearable Absorbent Article”; copending U.S. patent application Ser. No. 12/785,166 entitled “Insert With Advantageous Fastener Configurations And End Stiffness Characteristics For Two-Piece Wearable Absorbent Article”; copending U.S. patent application Ser. No. 12/785,181 entitled “Insert With Advantageous Fastener Configurations And End Stiffness Characteristics For Two-Piece Wearable Absorbent Article”; copending U.S. patent application Ser. No. 12/841,553 entitled “Two-Piece Wearable Absorbent Article With Advantageous Fastener Performance Configurations”; copending U.S. patent application Ser. No. 12/841,467 entitled “Flexible Reusable Outer Covers For Disposable Absorbent Inserts”; and copending U.S. patent application Ser. No. 12/841,600 entitled “High-Capacity Disposable Absorbent inserts For Reusable Outer Covers”, each of which is hereby incorporated by reference.

### Collection of Test Samples

For the Lateral Extensibility Test Method, use a test sample collected as follows. The test sample is collected from a reusable chassis. The reusable chassis includes all of the chassis structures and features, which are configured as reusable, but none of the chassis structures and features, which are not configured as reusable. For example, a machine washable cloth outer cover is considered part of a reusable chassis As another example, a washable liquid impermeable barrier that is configured to fit inside of the outer cover is also considered to be part of a reusable chassis. An absorbent insert is not considered to be part of a chassis, so an absorbent insert is not part of a reusable chassis.

If the reusable chassis includes a liquid impermeable barrier, then place the barrier into its in-use position with respect to the chassis. If the barrier is configured to attach to the chassis, then attach the barrier to the chassis, such that the barrier is fully and properly attached in its in-use position.

Align the front waist edge of the reusable chassis with the back waist edge of the reusable chassis while the portion of the longitudinal centerline in the front is overlying the portion of the longitudinal centerline in the back; this is the bifolded configuration. While maintaining the bifolded configuration, lay the chassis on a clean, smooth, flat, horizontal work surface (such as a table-top), so that the outside of the front of the chassis is facing upward.

Continue to maintain the bifolded configuration of the chassis by firmly holding down the front waist edge, such that the front waist edge holds the back waist edge against the work surface. While holding down the front waist edge, use one hand in a smoothing and pressing motion to slowly, continuously, and progressively flatten the chassis against the work surface, from the waist edge to the crotch portion of the chassis. Once the flattening is complete, continue to hold the crotch portion of the reusable chassis flat and mark the outside of the chassis at a point along the longitudinal centerline that is the farthest outer extent of the laterally oriented bifold in the crotch portion of the chassis.

If the reusable chassis includes a liquid impermeable barrier, then perform the following steps for marking the barrier. While continuing to hold the crotch portion flat, firmly hold down on a portion of the front of the chassis that overlies the barrier; this will hold the barrier in place in the bifolded configuration. While holding down the overlying portion of the front, release the hold on the front waist edge of the chassis. If the barrier is attached to the chassis, then, while holding down the overlying portion of the front, detach the barrier from the front of the chassis as far as possible (while maintaining the hold), but do not detach the barrier from the back of the chassis. Fold back a portion of the front of the chassis to expose the front edge of the barrier. While continuing to hold down the overlying portion of the front, firmly hold down the front edge of the barrier. Then release the hold on the overlying portion of the front of the chassis, Maintain the bifolded configuration of the barrier by continuing to firmly holding down the front edge of the barrier, such that the front edge holds the back of the barrier against the back of the chassis. Detach any remaining attachment between the barrier and the front of the chassis. Fold back the remainder of the front of the chassis to fully expose the front of the chassis facing side of the barrier. While holding down the front edge of the barrier, use one hand in a smoothing and pressing motion to slowly, continuously, and progressively flatten the barrier against the back of the chassis, from the front edge to the crotch portion of the barrier. Once the flattening is complete, continue to hold the crotch portion of the barrier flat and mark the chassis facing side of the barrier at a point along the longitudinal centerline that is the farthest outer extent of the laterally oriented bifold in the crotch portion of the barrier. After this, fully detach the barrier from the chassis and separate the barrier from the chassis for the cutting and marking described below.

Cut any leg elastics off of the reusable chassis, by cutting continuously all the way around the leg elastics, with the cut line positioned immediately adjacent to the elastics, in order to remove a minimal amount of chassis material. Lay the chassis out on a clean, smooth, flat, horizontal work surface (such as a table-top), no that the outside of the chassis is facing upward. Secure the front waist edge to the work surface, so that the front waist edge is straight and flat. While keeping the back waist edge parallel with the front waist edge (and keeping the longitudinal centerline of the chassis perpendicular to the waist edges) pull the back waist edge longitudinally away from the front waist edge, until wrinkles and slack are removed from the reusable chassis (but the chassis is not extended to a stretched condition); then secure the back waist edge to the work surface, to hold the chassis in this pulled flat state.

While the reusable chassis is secured in the pulled flat state, draw three lines on the outside of the chassis. First, draw a lateral axis, which is straight line drawn laterally across the outside of the chassis, through the point marked on the outside, as described above. Second draw a second line parallel to the lateral axis, longitudinally offset from the lateral axis by 51 millimeters (toward the front). Third draw a third line parallel to the lateral axis, longitudinally offset from the lateral axis by 51 millimeters (toward the back).

Create the chassis portion of the test sample by cutting continuously all the way through and all the way across the reusable chassis, along the second line and the third line (marked on the outside as described, above). The test sample includes all of the structures of the reusable chassis that are connected to the portion between the second line and the third line, including all layers of the chassis and any chassis features that are attached to the chassis between the second line and the third line. Remove the test sample from the remaining portions of the reusable chassis, keeping any separate layers together in the same relative position as in the uncut reusable chassis. If the reusable chassis does not include a liquid impermeable barrier, then the chassis portion by itself forms the test sample.

If the reusable chassis includes a liquid, impermeable barrier, then perform the following steps for creating the barrier portion of the test sample. If the reusable chassis includes a liquid impermeable barrier, then cut any leg elastics off of the barrier, by cutting continuously all the way around the leg elastics, with the cut line positioned immediately adjacent to the elastics, in order to remove a minimal amount of barrier material. Lay the barrier out on a clean, smooth, flat, horizontal work surface (such as a table-top), so that the chassis facing side of the barrier is facing upward, Secure the front edge of the barrier to the work surface, so that the front edge is straight and flat. While keeping the back edge of the barrier parallel with the front edge of the barrier (and keeping the longitudinal centerline of the barrier perpendicular to the front and back edges) pull the back edge longitudinally away from the front edge, until wrinkles and slack are removed from the barrier (but the barrier is not extended to a stretched condition); then secure the back edge to the work surface, to hold, the barrier in this pulled flat state. While the reusable barrier is secured in the pulled flat state, draw three lines on the chassis facing side of the barrier. First, draw a lateral axis, which is straight line drawn laterally across the chassis facing side of the barrier, through the point marked on the outside, as described above. Second draw a second, line parallel to the lateral axis, longitudinally offset from the lateral axis by 51 millimeters (toward the front). Third draw a third line parallel to the lateral axis, longitudinally offset from the lateral axis by 51 millimeters (toward the front). Create the barrier portion of the test sample by cutting continuously all the way through and all the way across the barrier, along the second line and the third line (marked on the chassis facing side as described above). The test sample includes all of the structures of the reusable barrier that are connected to the portion between the second line and the third line, including all layers of the barrier and any barrier features that are attached to the barrier between the second line and the third line. Remove the test sample from the remaining portions of the barrier, keeping any separate layers together in the same relative position as in the barrier.

If the reusable chassis includes a liquid impermeable barrier, then combine the chassis portion and the barrier portion as described below to form the test sample. Lay the chassis portion of the test sample out on a clean, smooth, flat, horizontal work surface (such as a table-top), so that the inside of the chassis is facing upward. Lay the barrier portion of the test sample on top of the chassis portion, with the chassis facing side of the barrier portion facing downward toward the inside of the chassis. Align the second line of the chassis portion with the second line of the barrier portion while the portion of the longitudinal centerline in the barrier portion is overlying the portion of the longitudinal centerline in the chassis portion. Keep the chassis portion and the barrier portion in this same relative position for use in the Lateral Extensibility Test Method.

### Lateral Extensibility Test Method

The Lateral Extensibility Test Method utilizes a commercial tensile tester (e.g., from Instron Engineering Corp. (Canton, Mass.), SINTECH-MTS Systems Corporation (Eden Prairie, Minn.) or equivalent) interfaced with a computer. The computer is used to control the test speed and other test parameters and for collecting, calculating, and reporting the data. The grips of the tensile tester (need more info, the height of the grip will impact the gage length—e.g.) are flat rubber faced grips 2.54 mm tall and at least 102 mm wide. Also, the grips should provide adequate force to ensure that the test sample does not slip during testing. The load cell of tensile tester is selected so that the tensile response from the test sample is between 25 percent and 75 percent of the capacity of the load cell used. The tensile tester is calibrated according to the manufacturer's instructions.

Use a test sample, which was collected as described in Collection of Test Samples section. The test is performed under laboratory conditions of 23° C.±2° C. and relative humidity of 50 percent±2 percent. The samples are conditioned at the laboratory conditions for 24 hours prior to testing.

The test sample is mounted in the flats of the grips of the tensile tester as described below. All of the layers of the test sample are held between the upper grip and the lower grip of the tensile tester. The test sample is held by the grips, such that the lateral direction of the reusable chassis is parallel to the gauge length direction of the tensile tester. When closed, the inner face of each grip is parallel with the longitudinal direction of the reusable chassis and each grip holds the test sample continuously from the second line to the third line.

if the test sample includes a barrier portion, then the laterally innermost portion of one side of the barrier portion must extend 5-10 millimeters above the inner face of the upper grip when the upper grip is closed, and the laterally innermost portion of the other side of the barrier portion must extend 5-10 millimeters below the inner face of the lower grip when the lower grip is closed.

Position the test sample with respect to the upper grip, as described above then close the upper grip onto the test sample. Set the gauge length for the test sample, as described below. While the test sample is held by the upper grip, but before the test sample is held by the lower grip, allow the test sample to hang down. Move the grips apart to the largest distance wherein the test sample is positioned with respect to the lower grip, as described above, and wherein all slack is removed from the test sample, but the test sample is not extended, then close the lower grip onto the test sample. This distance between the bottom edge of the upper grip faces and the upper edge of the bottom grip faces is the gauge length. Record the gauge length.

Use the tensile tester to extend the test sample at a constant cross head speed of 100 millimeters per minute to a strain of 50 percent. For example, for a gauge length of 100 millimeters, a strain of 50 percent represents an extension of 50 millimeters to an extended length of 150 millimeters.

The computer data system records the force exerted on the test sample during the extension. From the resulting data generated, report the lateral extensibility of the body zone as Newtons of force (at 50 percent strain) divided by the width of the test sample (102 millimeters), which is all divided by the strain. That is:

$$Lateral Extensibility = \frac{( Force at 50 percent strain / Width of Test Sample )}{0.50}$$

Report lateral extensibility in units of Newtons per millimeter. When calculating lateral extensibility, do not take into account any other dimensions (such as thickness) of the test sample.

If enough articles are available, then repeat the Lateral Extensibility Test Method for three test samples. If not enough articles are available, then repeat the Lateral Extensibility Test Method for as many test samples as can be made from available articles. Take the average of the test sample values for Lateral Extensibility.

### Ear Extensibility Test Method

The Ear Extensibility Test Method utilizes a commercial tensile tester interfaced with a computer. The grips of the tensile tester must have flat surfaces and be 50 mm wide. Also, the grips should provide adequate force to ensure that the test sample does not slip during testing. The load cell of tensile tester is selected so that the tensile response from the test sample is between 25 percent and 75 percent of the capacity of the load cell used. The tensile tester is calibrated according to the manufacturer's instructions.

The tests are performed under laboratory conditions of 23° C.±2° C. and relative humidity of 50 percent±2 percent. The samples are conditioned at the laboratory conditions for 24 hours prior to testing.

To test the lateral extensibility of the ears in the front waist zone or the back waist zone, use a test sample as follows. The test sample is a reusable chassis. The reusable chassis includes all of the chassis structures and features, which are configured as reusable, but none of the chassis structures and features, which are not configured as reusable.

The test sample is mounted in the flats of the grips of the tensile tester as described below. The test sample is held by the grips, such that the lateral direction of the body zone of the reusable chassis is parallel to the gauge length direction of the tensile tester. When closed, the inner face of each grip is parallel with the longitudinal direction of the reusable chassis and each grip holds the test sample continuously across the grip width.

Position the test sample with respect to the grips, as described below then close the grips onto the test sample. If the ears have fasteners, then position the grips on the fasteners, such that the inner faces of the grips are aligned with the farthest laterally inboard points on the fasteners, and the fasteners are longitudinally centered on the grips. If the ears do not have fasteners then position the grips on the farthest laterally outboard portions of the ears, where each grip can hold the test sample continuously across the grip width.

While the test sample is held by the upper grip, but before the test sample is held by the lower grip, allow the test sample to hang down. Move the grips apart to the distance wherein the test sample is positioned with respect to the lower grip, as described above, and wherein all slack is removed from the test sample, but the test sample is not extended, then close the lower grip onto the test sample.

Use the tensile tester to extend the test sample by 200 millimeters at a constant cross head speed of 100 millimeters per minute. The computer data system records the force exerted on the test sample during the extension. From the resulting data generated, report the extensibility of the ears as Newtons of force (at 200 millimeters of extension).

If enough articles are available, then repeat the Ear Extensibility Test Method for three test samples. If not enough articles are available for three test samples, then repeat the Ear Extensibility Test Method for as many test samples as can be made from available articles. Take the average of the test sample values for Ear Extensibility.

### Dimension Method

Various dimensions and ratios thereof are specified herein. Each dimension is measured according to the following method. All testing is performed in a conditioned room maintained at about 23 C±2 C and about 50 percent±2 percent relative humidity. Herein, width and length of the specimen are a lateral width and longitudinal length as defined herein. Precondition specimens at about 23 C±2 C and about 50 percent±2 percent relative humidity for 2 hours prior to testing.

Prepare the article for testing as follows:

1. Cut any leg elastics off of the reusable chassis, by cutting continuously all the way around the leg elastics, with the cut line positioned immediately adjacent to the elastics, in order to remove a minimal amount of chassis material.

2. Lay the article on a substantially flat, horizontal surface.

3. Secure the article to the surface such that all process-induced contraction is pulled out. The article is secured to the flat, horizontal surface with clamps or adhesive tape capable of holding the absorbent core assembly with process-induced contraction pulled out.

4. Identify points between which widths and/or lengths of a portion the article is to be measured, per definitions contained herein.

5. Measure each needed dimension to the nearest 1 mm using a steel ruler traceable to NIST.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross-referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended that the appended claims cover all such changes and modifications, and that nothing in the foregoing description or the figures, but rather, only the appended claims, limit the scope of the invention.

What is claimed is:

1. A reusable chassis, configured for use with a disposable absorbent insert as a wearable absorbent article, the reusable chassis comprising:
a lateral axis;
a front waist edge and a front waist zone, proximate to the front waist edge;
a rear waist edge and a rear waist zone, proximate to the rear waist edge; and
a reusable chassis overall longitudinal length, measured longitudinally between the front waist edge and the rear waist edge;
leg bands;
a body zone, disposed longitudinally between the front waist zone and the rear waist zone, disposed laterally inboard to the leg bands, and overlapping the lateral axis;
wherein, when the chassis is laid out flat, a narrowest lateral width of the reusable chassis is greater than or equal to 20 percent of the overall longitudinal length but less than or equal to 60 percent of the overall longitudinal length; and
wherein the body zone has a lateral extensibility that is greater than or equal to 0.03 Newtons per millimeter but less than or equal to 0.24 Newtons per millimeter, when tested according to the Lateral Extensibility Test Method.

2. The reusable chassis of claim 1, wherein, the chassis is laid out flat, a narrowest lateral width of the reusable chassis is greater than or equal to 23 percent of the overall longitudinal length but less than or equal to 52 percent of the overall longitudinal length.

3. The reusable chassis of claim 1, wherein, the chassis is laid out flat, a narrowest lateral width of the reusable chassis is greater than or equal to 26 percent of the overall longitudinal length but less than or equal to 44 percent of the overall longitudinal length.

4. The reusable chassis of claim 1, wherein, the chassis is laid out flat, a narrowest lateral width of the reusable chassis is greater than or equal to 29 percent of the overall longitudinal length but less than or equal to 36 percent of the overall longitudinal length.

5. The reusable chassis of claim 1, wherein the body zone has a lateral extensibility that is greater than or equal to 0.07 Newtons per millimeter but less than or equal to 0.22 Newtons per millimeter, when tested according to the Lateral Extensibility Test Method.

6. The reusable chassis of claim 1, wherein the body zone has a lateral extensibility that is greater than or equal to 0.11 Newtons per millimeter but less than or equal to 0.20 Newtons per millimeter, when tested according to the Lateral Extensibility Test Method.

7. The reusable chassis of claim 1, wherein the body zone has a lateral extensibility that is greater than or equal to 0.15 Newtons per millimeter but less than or equal to 0.18 Newtons per millimeter, when tested according to the Lateral Extensibility Test Method.

8. The reusable chassis of claim 1, wherein the body zone has an overall longitudinal length that is greater than or equal to 30 percent of the overall longitudinal length but less than or equal to 70 percent of the overall longitudinal length.

9. The reusable chassis of claim 1, wherein the body zone has an overall longitudinal length that is greater than or equal to 40 percent of the overall longitudinal length but less than or equal to 60 percent of the overall longitudinal length.

10. The reusable chassis of claim 1, wherein the front waist zone has a lateral extensibility that is greater than or equal to 4 Newtons but less than or equal to 16 Newtons, when tested according to the Ear Extensibility Test Method.

11. The reusable chassis of claim 1, wherein the front waist zone has a lateral extensibility that is greater than or equal to 6 Newtons but less than or equal to 14 Newtons, when tested according to the Ear Extensibility Test Method.

12. The reusable chassis of claim 1, wherein the front waist zone has a lateral extensibility that is greater than or equal to 8 Newtons but less than or equal to 12 Newtons, when tested according to the Ear Extensibility Test Method.

13. The reusable chassis of claim 1, wherein the front waist zone is adjacent to the front waist edge.

14. The reusable chassis of claim 1, wherein the rear waist zone has a lateral extensibility that is greater than or equal to 4 Newtons but less than or equal to 16 Newtons, when tested according to the Ear Extensibility Test Method.

15. The reusable chassis of claim 1, wherein the rear waist zone has a lateral extensibility that is greater than or equal to 6 Newtons but less than or equal to 14 Newtons, when tested according to the Ear Extensibility Test Method.

16. The reusable chassis of claim 1, wherein the rear waist zone has a lateral extensibility that is greater than or equal to 8 Newtons but less than or equal to 12 Newtons, when tested according to the Ear Extensibility Test Method.

17. The reusable chassis of claim 1, wherein the rear waist zone is adjacent to the rear waist edge.

18. A wearable absorbent article, comprising the reusable chassis of claim 1 and an absorbent insert.

19. The wearable absorbent article of claim 18, wherein the absorbent insert is a disposable absorbent insert.

20. The wearable absorbent article of claim 19, wherein the disposable absorbent insert includes superabsorbent particles.
